(19) United States
(12) Patent Application Publication
(10) Pub. No.: US 2005/0129677 A1
(43) Pub. Date: Jun. 16, 2005

(54) Lipid rafts and clostridial toxins

(76) Inventors: Shengwen Li, Irvine, CA (US); Kei Roger Aoki, Coto de Caza, CA (US)

Correspondence Address: COZEN O'CONNOR, P.C., 1900 MARKET STREET, PHILADELPHIA, PA, 19103-3508, US

(21) Appl. No.: 10/732,703

(22) Filed: Dec. 10, 2003

(51) Int. Cl. A61K 39/395

(52) U.S. Cl. 424/130.1

(57) ABSTRACT

The present invention is directed to methods of altering the degree of internalization of a Clostridial toxin; methods of preventing or treating botulinum toxin intoxication; methods of treating metabolic disorders, muscular disorders, nervous system disorders, and/or pain conditions; methods of inhibiting the formation of lipid rafts on cell membranes; methods of treating a disease associated with lipid rafts; and methods of identifying a compound that alters the internalization of a Clostridial toxin.

### FIELD OF THE INVENTION

The present invention is directed to methods of altering the degree of internalization of a Clostridial toxin; methods of preventing or treating botulinum toxin intoxication; methods of treating metabolic disorders, muscular disorders, nervous system disorders, and/or pain conditions; methods of inhibiting the formation of lipid rafts on cell membranes; methods of treating a disease associated with lipid rafts; and methods of identifying a compound that alters the internalization of a Clostridial toxin.

### BACKGROUND OF THE INVENTION

Botulinum toxins have been used in clinical settings for the treatment of neuromuscular disorders characterized by hyperactive skeletal muscles. In 1989, a botulinum toxin type A complex has been approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

It has been reported that botulinum toxin type A has been used in clinical settings as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U.

Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Jyons, J., et al., Tremor-Predominant Parkinson's Disease, Drugs & Aging 16(4);273-278:2000.

It is known that botulinum toxin type A can have an efficacy for up to 12 months (European J Neurology 6 (Supp 4): S111-S1150:1999), and in some circumstances for as long as 27 months. The Laryngoscope 109:1344-1346:1999. However, the usual duration of an intramuscular injection of Botox® is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

In addition to having pharmacologic actions at the peripheral location, botulinum toxins may also have inhibitory effects in the central nervous system. Work by Weigand et al, Nauny-Schmiedeberg's Arch. Pharmacol. 1976; 292, 161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

A botulinum toxin has also been proposed for the treatment of rhinorrhea, hyperhydrosis and other disorders mediated by the autonomic nervous system (U.S. Pat. No. 5,766,605), tension headache (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319), various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977). Additionally, controlled release toxin implants are known (see e.g. U.S. Pat. Nos. 6,306,423 and 6,312,708).

Seven generally immunologically distinct botulinum neurotoxins have been characterized: botulinum neurotoxin serotypes A, B, C₁, D, E, F and G. These serotypes are distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotypes A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem, J 1;339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacteria as complexes comprising the 150 kD botulinum toxin protein molecule along with associated non-toxin proteins. Thus, the botulinum toxin type A complex can be produced by Clostridial bacteria as 900 kD, 500 kD and 300 kD forms. Botulinum toxin types B and C₁ is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemagglutinin protein and a non-toxin and non-toxic nonhemagglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897). Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [3H]Noradrenaline and [³H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) are dependent, at least in part, upon the three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48:249-53:1997.

Ganglioside molecules are a class of glycosphingolipids that comprise a high percentage of the plasma membrane of neuronal cells; gangliosides were the first membrane components found to have BoNT and TeNT binding activity. Subsequent studies identified gangliosides of series b, especially GT1b and GD1b, to have the highest affinity for these Clostridial toxins. Currently, a two-receptor model is favored, which proposes non-specific affinity-mediated binding of the toxins to gangliosides in the plasma membrane, as well as an additional, more specific engagement of each toxin with its own particular protein receptor. Synaptotagmin in combination with gangliosides has been shown to act as a receptor for BoNT/A, BoNT/B, and BoNT/E. The Clostridium perfringens toxin has also been demonstrated to form a heptameric channel through the plasma membrane, allowing entry of the toxin into cells. A putative receptor for TeNT has also been described, although the protein(s) involved remain uncharacterized at the molecular level.

The historical “fluid mosaic” model of a lipid bilayer plasma membrane is well known. More recently, “liquid-ordered” membrane sites, with microdomains enriched in cholesterol, sphingolipids, and glycosphingolipids, have been the focus of investigations into plasma membrane mechanics. These microdomains, also known as lipid rafts, detergent-insoluble glycolipid-rich domains (DIGs), caveolae-like detergent-insoluble membrane microdomains, glycosphingolipid signaling domains (GSD), glycolipid-enriched membranes (GEMs), and low-density Triton-insoluble (LDTI) complexes, are believed to play a central role in signal transduction and protein trafficking. High concentrations of several signaling molecules, including inositol 1,4,5 triphosphate receptors, protein kinase C, G protein-coupled receptors, multiple heterotrimeric GTP-binding proteins, non-receptor tyrosine kinases, ATP-dependent calcium-pump proteins, endothelial nitroxide synthase (eNOS), and epidermal growth factor (EGF) receptors, have been found associated with lipid rafts.

As discussed, botulinum toxin is an effective therapeutic in the prevention and treatment of a number of medical conditions.

Thus, there is a continued need to have more effective drugs and methods for treating botulism. Additionally, there is a continued need to have more effective use of botulinum toxins to treat medical conditions. The present invention provides for such improvements.

### SUMMARY OF THE INVENTION

The present invention provides for effective methods of altering the degree of internalization of a Clostridial toxin into a cell. In some embodiments, the method comprises the step of altering the activity of lipid rafts or caveolae on a membrane of a cell.

Further in accordance with the present invention, the activity of the lipid rafts may be decreased by contacting the membrane of a cell with an activity inhibitor, such as an antibody or a lipid raft concentration inhibitor.

Still further in accordance with the present invention, the activity of lipid rafts may be increased by contacting the membrane of a cell with a lipid raft activity enhancer. In some embodiments, an activity enhancer comprises an antibody. In some embodiments, an activity enhancer comprises a lipid raft concentration enhancer, such as a cholesterol-enhancing agent and a sphingolipid-enhancing agent.

Still further in accordance with the present invention, a method of preventing or treating botulinum intoxication in a mammal is provided. In some embodiments, the method comprises the step of administering a lipid raft activity inhibitor to the mammal to prevent or to treat botulinum intoxication. In some embodiments, the lipid raft activity inhibitor comprises an antibody or cholesterol-reducing agent, a sphingolipid-reducing agent or combinations thereof.

Still further in accordance with the present invention, methods of preventing or treating a metabolic disorder, a muscular condition, a nervous system disorder and/or a pain condition in a mammal are provided. In some embodiments, the methods comprise the step of administering a lipid raft activity enhancer, and administering a Clostridial toxin.

Still further in accordance with the present invention, methods of inhibiting the formation of lipid rafts on a cell are provided. In some embodiments, the methods comprise the step of contacting the cell with a Clostridial toxin, for example botulinum toxin to inhibit the formation of lipid rafts on a cell membrane.

Still further in accordance with the present invention, methods of treating a disease associated with lipid rafts are provided. The methods comprise a step of administering a Clostridial toxin to a mammal. Non-limiting examples of diseases associated with lipid rafts include hepatic insulin resistance, obesity, diabetes, hematopoietic condition, and immunoinflammatory condition.

Still further in accordance with the present invention, methods of identifying a compound that alters internalization of a Clostridial toxin into a cell are provided. In some embodiments, the methods comprise the steps of contacting a cell sensitive to Clostridial toxin with a test compound, and screening for compounds that alter the affinity of the Clostridial toxin for lipid rafts.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

Definitions

“Lipid rafts” (also known as liquid-ordered domains, membrane microdomains, and detergent-insoluble glycolipid-rich domains) are assemblies of sphingolipids and cholesterol in the exoplasmic leaflet of the fluid bilayer probably interacting with the underlying cytosolic leaflet. These assemblies function as platforms in membrane trafficking and signaling. A number of proteins specifically interact with rafts and these can be identified by biochemistry and mass spectrometry. Lipid rafts are small, around 50-100 nanometers in diameter. Some lipid rafts comprise caveolin family members (e.g., caveolin and/or flottillin, which are proteins). In some embodiments, caveolin-containing lipid rafts may contain a caveolin family member selected from the group consisting of caveolin-1alpha, caveolin-1beta, caveolin-2, caveolin-3, flottillin-1, flottillin-2 and combinations thereof. Moreover, the concentration of caveolin family member may vary according to cell type. Caveolin family members are also known to be differentially expressed, and some caveolins are specifically expressed in neuronal cells, astrocytes, glial cells, striated muscle cells, smooth muscle cells, cardiac cells, adipocytes, endothelial cells, secretory cells, type I pneumocytes, lung cells, kidney cells, dendritic cells, Mast cells, macrophages, T-cells, and B-cells.

“Caveolae” are specialized lipid rafts that perform a number of signaling functions. Caveolae are 50-100 nm “flask shaped” invaginations of the plasma-membrane. They are found in a variety of cell types, especially endothelial cells. Many proteins and lipids are known to be enriched in caveolae.

“Activity of lipid rafts” are, for example, cellular activities that coordinated by lipid rafts. A wide range of activities is believed to be coordinated by lipid rafts. Lipid rafts are primarily known to be associated with cellular trafficking of molecules, by both endocytosis and exocytosis. Lipid rafts serve as regions for the assembly of vesicle fusion proteins during exocytosis of molecules from neurons or secretory cells. Furthermore, cellular and/or exogenous molecules that interact with lipid rafts can use them as transport shuttles. Lipid rafts can act as molecular sorting machines that coordinate the inclusion of signaling molecules into cellular membranes, thereby serving as platforms or recognition points for the assembly of receptor/ligand complexes. Thus, lipid rafts may act as spatiotemporal organizers of signal transduction pathways within selected cells or subcellular areas. Lipid rafts can also mediate conformational changes in the membrane, allowing entry or exit of molecules from cells. Additionally, lipid rafts can act as sites for the localization, compartmentalization and/or concentration of molecules, serving as points of entry for pathogens or toxin proteins.

The term “botulinum toxin intoxication” means a condition caused by one or more of the seven serotypes of active botulinum toxins usually produced by Clostridium botulinum. The symptoms of botulinum toxin intoxication include acute symmetric, descending flaccid paralysis with prominent bulbar palsies, typically presenting within 12 to 72 hours after exposure. Botulinum toxin intoxication may be fatal if it is not properly treated.

A “diseases associated with a formation of a lipid raft or a caveolae” are diseases wherein the inhibition of lipid raft formation or inhibition/regulation of caveolae formation would alleviate the symptoms of the disease or treat the disease.

The term “heavy chain” means the heavy chain of a botulinum toxin. It has a molecular weight of about 100 kDa and can be referred to herein as heavy chain or as H.

The term “H_{N}” means a fragment (having a molecular weight of about 50 kDa) derived from the Heavy chain of a botulinum toxin, which is approximately equivalent to the amino terminal segment of the Heavy chain, or the portion corresponding to that fragment in the intact Heavy chain. It is believed to contain the portion of the natural or wild type botulinum toxin involved in the translocation of the light chain across an intracellular endosomal membrane.

The term “H_{C}” means a fragment (about 50 kDa) derived from the Heavy chain of a botulinum toxin which is approximately equivalent to the carboxyl terminal segment of the Heavy chain, or the portion corresponding to that fragment in the intact Heavy chain.

The term “light chain” means the light chain of a botulinum toxin. It has a molecular weight of about 50 kDa, and can be referred to as light chain, L or as the proteolytic domain (amino acid sequence) of a botulinum toxin. The light chain is believed to be effective as an inhibitor of exocytosis, including as an inhibitor of neurotransmitter (i.e. acetylcholine) release when the light chain is present in the cytoplasm of a target cell.

The term “targeting moiety” means a molecule that is recognized by and binds to a receptor on a surface of a cell, preferably to a specific type of cell.

The term “mammal” as used herein includes, for example, humans, rats, rabbits, mice and dogs.

The term “local administration” means direct administration by a non-systemic route at or in the vicinity of the site of an affliction, disorder or perceived pain.

### DESCRIPTION OF EMBODIMENTS

The present invention is base, in part, upon the discovery that the degree of internalization of a Clostridial toxin into a cell may be altered by altering the activity of lipid rafts on a membrane of a cell. This discovery has significant medical implications and uses. For example, the degree of internalization of Clostridial toxin may be decrease by decreasing the activity of lipid rafts—effectively treating or inhibiting Clostridial toxin intoxication. As discussed, the use of Clostridial toxins is effective in treating many medical conditions. Thus, in some embodiments, it would be advantageous to increase the activity of lipid rafts, so that there would be an increased internalization of Clostridial toxin that is administered.

The present invention is also based, in part, upon the discovery that a Clostridial toxin may inhibit the formation of lipid rafts on a cell. This discovery has significant medical implication and uses. For example, based on the present discovery, a Clostridial toxin may be administered to treat various medical conditions associated with lipid raft formations.

Novel methods that employ the referenced Clostridial toxins herein may also employ any toxin produced by Clostridium beratti, Clostridium butyricum, Clostridium tetani bacterium or Clostridium botulinum. In some embodiments, the Clostridial toxin is a toxin is selected from the group consisting of: botulinum toxin types A, B, C₁, D, E, F and G. In some embodiments, the Clostridial toxin is botulinum toxin type A. Accordingly, it is possible that any toxin produced by Clostridium beratti, Clostridium butyricum, Clostridium tetani bacterium, Clostridium botulinum, botulinum toxin types A, B, C₁, D, E, F or G may be used where the use of Clostridial toxin is referenced.

I. Methods of altering the degree of internalization of a Clostridial toxin into a cell: The degree of internalization of the Clostridial toxin may be altered by altering the activity of the lipid rafts (e.g. caveolae) on the membrane of a cell. For example, the activity lipid rafts (e.g. caveolae) may be decreased by contacting the membrane of a cell with an activity inhibitor. Non-limiting examples of activity inhibitors include an antibody, such as humanized antibodies, polyclonal antibodies, monoclonal antibodies, and function blocking antibodies.

Antibodies may serve as an activity inhibitor by binding to and physically inhibiting, interfering with or blocking the function of a critical component of lipid rafts. This component may be an integral or membrane-anchored protein component, a cholesterol, or a sphingolipid (including a ganglioside). The binding of antibodies may change the conformation of the critical component within the membrane context of the lipid raft, resulting in an inhibition of the component's function. The binding of antibodies to the critical lipid raft component may physically inactivate the component or directly prevent it from participating in a signal transduction event. The binding of antibodies may cause an aggregation of multiple components, disrupting their localization and/or concentration within lipid rafts.

In some embodiments, antibodies that are effective to inhibit the activity of lipid rafts (e.g. caveolae) may be targeted against components associated with lipid rafts. Non-limiting examples of components associated with lipid rafts include: caveolin-1, caveolin-2, caveolin-3, flotillin-1, flotillin-2, reggie-1, reggie-2, stomatin, VIP36, LAT/PAG, MAL, BENE, syntaxin-1, syntaxin-4, synapsin I, adducin, VAMP2, VAMP/synaptobrevin, synaptobrevin II, SNARE proteins, SNAP-25, SNAP-23, a membrane-associated Clostridial toxin receptor protein, synaptotagmin I, synaptotagmin II or GPI-anchored proteins. Antibodies which recognize gangliosides such as GM1, GD1a, GD1b, GQ1b and GT1b may also be used to decrease the activity of lipid rafts.

In some embodiments, an antibody against a caveolin (e.g., caveolin-1, caveolin-2, caveolin-3) is employed to inhibit the activity of lipid rafts (e.g., caveolae). In some embodiments, the antibody against a caveolin may be conjugated with a transporter to transport the caveolin into the cell. Various transporters are known in the art. For example U.S. Pat. No. 6,203,794 teaches the use of an inactive Clostridial toxin as a transporter. (The disclosure of the U.S. Pat. No. 6,203,794 is incorporated in its entirety herein by reference). In some embodiments, an antibody against a caveolin is conjugated to an inactive Clostridial toxin as is taught by the U.S. Pat. No. 6,203,794. Hereinafter, a an antibody against a caveolin conjugated to an inactive Clostridial toxin is referred to as a anti-caveolin conjugate.

In some embodiments, the anti-caveolin conjugate comprises an antibody against a caveolin and an inactive botulinum toxin. For example, an anti-caveolin conjugate may comprise an antibody against a caveolin-1 and an inactive botulinum toxin type A, or an antibody against a caveolin-2 and an inactive botulinum toxin type A, or an antibody against a caveolin-3 and an inactive botulinum toxin type A. In some embodiments, anti-caveolin conjugate comprises an antibody against a flottillin and an inactive botulinum toxin. For example, an anti-caveolin conjugate may comprise an antibody against a flottillin-1 and an inactive botulinum toxin type A, or an antibody against a flottillin-2 and an inactive botulinum toxin type A.

Without wishing to limit the invention to any theory or mechanism of operation, it is believed that the activity of lipid rafts (e.g. caveolae) may also be altered by altering the concentration of lipid rafts. The alteration in the concentration of lipid rafts alters the activity of lipid rafts because, for example, a reduction in the concentration of lipid rafts within a cell membrane decreases the availability of docking sites or access points for lipid raft-interacting molecules such as toxins, pathogens, signal transduction molecules, extracellular ligands for receptors, caveolins and SNAP and SNARE fusion complex proteins. A decrease in availability of docking sites or access points can lead to a reduction in endocytic and/or exocytic vesicle fusion and a disruption of membrane trafficking. On the other hand, an increase in the concentration of lipid rafts within a membrane increases the availability of the docking sites or access points for lipid raft-interacting molecules such as toxins, pathogens, signal transduction molecules, extracellular ligands for receptors, caveolins and SNAP and SNARE fusion complex proteins. An increase in availability of docking sites or access points can lead to an enhancement of endocytic and/or exocytic vesicle fusion and an enhancement of membrane trafficking.

In some embodiments, the activity of lipid rafts (e.g. caveolae) may be decreased by contacting the membrane of a cell with a lipid raft activity inhibitor that causes a change in concentration of the lipid rafts. Thus, a lipid raft (e.g. caveolae) activity inhibitor may include a lipid raft concentration inhibitor. Non-limiting examples of lipid raft (e.g. caveolae) concentration inhibitors include: cholesterol-reducing agents or sphingolipid-reducing agents. Non-limiting examples of cholesterol-reducing agents include statins, cyclodextrin, saponin, and filipin. Non-limiting examples of sphingolipid-reducing agents include synthetic sphingolipid analogues and inhibitors of sphingolipid synthesis (e.g., L-cycloserine, fumonisin B1, and D-threo-1-phenyl-2-decanoylamino-3-morpholino-1-propanol).

The cholesterol-reducing agents or sphingolipid-reducing agents may inhibit or decrease the concentration of lipid rafts by preventing the formation of lipid rafts, or affecting their composition and/or activity. It is known that the local lipid environment has a significant impact on the formation, composition and activity of lipid rafts. For example, caveolin proteins on the cytosolic leaflet of the plasma membrane are known to interact with cholesterol. In fact, there is evidence for a role of caveolin in transfer of cholesterol into lipid raft domains of the plasma membrane. Caveolin knockout mice have altered lipid homeostasis suggesting that the predicted role of caveolin proteins in transferring cholesterol into lipid rafts significantly affects lipid raft composition. Thus, agents which reduce the levels of cholesterol and sphingolipid components of lipid rafts are predicted to affect the formation, composition and/or activity of lipid rafts. Furthermore, changing the composition of lipid rafts may result in a change in specificity of lipid raft-interacting molecules such as botulinum toxin for certain neurons.

In some embodiments, the activity of lipid rafts (e.g. caveolae) may also be increased. For example, the activity of lipid rafts (e.g. caveolae) may be increased by contacting the membrane of a cell with a lipid raft activity enhancer. Non-limiting examples of lipid raft (e.g. caveolae) activity enhancer include an antibody or a lipid raft concentration enhancer. Without wishing to limit the invention to any theory or mechanism of operation, an antibody may act as a lipid raft activity enhancer by linking together (or causing colocalization or clustering of) components of lipid rafts. This linking together results in an increase of lipid raft activity because some components of lipid rafts, such as transmembrane proteins, growth factor receptors and other signal transduction proteins are known to be activated by dimerization. Furthermore, it is known that the activation of multimeric complexes can be regulated by the assembly state of the complex. Thus, the colocalization or clustering of components of a multimeric complex within lipid rafts may regulate the activity of the multimeric complex, thereby having an effect on the activity of lipid rafts. Additionally, several proteins are also known to be regulated by posttranslational modifications such as phosphorylation, acetylation, palmitoylation and ubiquitination; by being brought into contact with an interacting protein, a critical protein component of lipid rafts may be posttranslationally modified and activated, thereby increasing the activity of the lipid raft.

In some embodiments, a lipid raft (e.g. caveolae) concentration enhancer may be a cholesterol-enhancing agent or a sphingolipid-enhancing agent. Gangliosides and GPI-anchored proteins are non-limiting examples of the sphingolipid and protein components, respectively, sometimes found in lipid rafts. In living cells, GPI-anchored proteins have been shown to be clustered within lipid rafts, and this clustering was dependent on the level of cholesterol in the cell. Exogenous application of gangliosides to living cells has been demonstrated to affect the properties of lipid rafts, abolishing clustering of GPI-anchored proteins and displacing them from lipid rafts (Simons, et al., 1999 Mol. Biol. Cell 10(10): 3187-3196). Thus, changing the concentration of one components of lipid rafts can have far-reaching effects on other components comprising lipid rafts. In some embodiments of the present invention, a lipid raft (e.g. caveolae) concentration enhancer may be a cholesterol-enhancing agent or a sphingolipid-enhancing agent. A non-limiting example a of cholesterol-enhancing agent is a synthetic cholesterol analogue such as 313-chlorocholestene, a nonfusogenic analogue of cholesterol. An example of a sphingolipid-enhancing agents is a synthetic sphingolipid analogue such as the C₆-NBD-labeled sphingolipids C₆-NBD-glucosylceramide and C₆-NBD-sphingomyelin (van IJzendoom and Hoekstra, 1999 Mol. Biol. Cell 10(10): 3449-3461).

The cholesterol-enhancing agent and the sphingolipid-enhancing agent enhance the lipid raft activity by replacing cholesterol and sphingolipids (including gangliosides), respectively, in lipid rafts within the plasma membrane, thus changing the composition of lipid rafts. Such a change in lipid raft composition can enhance or diminish the function of lipid rafts. If the analogue has the same activity as the cholesterol or sphingolipid component it replaces, an increased concentration of the analogue would act to increase the activity of the lipid rafts. If the analogue has a diminished activity as compared to the cholesterol or sphingolipid component it replaces, treatment with the analogue would reduce lipid raft activity.

In some embodiments, the degree of internalization of a Clostridial toxin into a cell may be altered by changing the concentration of caveolin family members (caveolin-1alpha, caveolin-1beta, caveolin-2, caveolin-3, flottillin-1, flottilli-2, etc.) on a membrane of the cell. The change in the concentration of caveolin may alter the degree of internalization of a Clostridial toxin because the Clostridial toxin, e.g., botulinum toxin, is believed to directly or indirectly interact with caveolin proteins, and caveolin-containing lipid rafts are believed to mediate the endocytosis of Botulinum toxin as well as the exocytosis of vesicles involved in secretion and release of neurotransmitters. For example, the degree of internalization of Clostridial toxin may be reduced by decreasing the concentration of caveolin proteins in a cell membrane. In some embodiments, a method of decreasing the concentration of caveolin proteins is to contact the membrane of a cell with antibodies. Non-limiting examples of antibodies that may be employed include humanized antibodies, polyclonal antibodies, monoclonal antibodies, and function blocking antibodies.

Antibodies that decrease the concentration of caveolin proteins in a cell membrane may be targeted against caveolin-1alpha, caveolin-1beta, caveolin-2, caveolin-3, flotillin-1, flotillin-2, reggie-1, reggie-2, stomatin, VIP36, LAT/PAG, MAL, BENE, syntaxin-1, syntaxin-4, synapsin I, adducin, VAMP2, VAMP/synaptobrevin, synaptobrevin II, SNARE proteins, SNAP-25, SNAP-23, a membrane-associated Clostridial toxin receptor protein, synaptotagmin I, synaptotagmin II and GPI-anchored proteins. Antibodies which recognize gangliosides such as GM1, GD1a, GD1b, GQ1b and GT1b may also be used to decrease the concentration of caveolin proteins.

One of ordinary skill in the art would know how to prepare these antibodies. For example, one method for the production of antibodies commonly known in the art is to express and purify a recombinant peptide or protein of interest using standard molecular biological techniques, and then inject this purified peptide or protein into a mammal, such as a rabbit or a mouse. After an adequate period of time and multiple immunizations, the immune sera is then obtained from the injected animal, and, using affinity purification methods, antibodies with enhanced specificity for the particular peptide or protein of interest can be further purified and isolated from the complex mixture of the immune sera. Anti-ganglioside antibodies can also be generated, as can antibodies targeted against other non-peptide molecules (see Schwerer, et al., 1999 Infect. Immun. 67(5):2414-2420). Other methods of antibody production, such as the production of monoclonal antibodies are also well known in the art.

In some embodiments, any antibody identified herein may be conjugated with a transporter to transport the antibody into a cell. In some embodiments, the antibodies are conjugated with an inactive Clostridial toxin as taught by Dolly in U.S. Pat. No. 6,203,794, to form a conjugate that may be transported into a cell. For example, such conjugates may comprise one or more of the following antibodies against caveolin-1alpha, caveolin-1beta, caveolin-2, caveolin-3, flotillin-1, flotillin-2, reggie-1, reggie-2, stomatin, VIP36, LAT/PAG, MAL, BENE, syntaxin-1, syntaxin-4, synapsin I, adducin, VAMP2, VAMP/synaptobrevin, synaptobrevin II, SNARE proteins, SNAP-25, SNAP-23, a membrane-associated Clostridial toxin receptor protein, synaptotagmin I, synaptotagmin II, GPI-anchored proteins, GM1, GD1a, GD1b, GQ1b and/or GT1b.

In some embodiments, the concentration of caveolin proteins may also be decreased by contacting the membrane of a cell with a cholesterol-reducing agent or a sphingolipid-reducing agent described above.

In some embodiments, the concentration of caveolin proteins may be increased by contacting the membrane of a cell with a cholesterol-enhancing agent or a sphingolipid-enhancing agent, such as synthetic cholesterol or sphingolipid analogues as described above.

In some embodiments, the concentration of caveolin protein may be increased by stimulating caveolin gene expression.

II. Methods of preventing or treating Clostridial toxin intoxication in a mammal: A lipid raft activity inhibitor causes a decrease in the internalization of Clostridial toxin, for example botulinum toxin, into cells. As such, the lipid raft activity inhibitor is effective in treating Clostridial toxin intoxication. In some embodiments, the method treating or preventing Clostridial toxin intoxication, for example botulinum toxin intoxication, comprises the step of administering to a mammal in need thereof a lipid raft (e.g. caveolae) activity inhibitor. As described above, non-limiting lipid raft activity inhibitors include an antibody, a cholesterol-reducing agent, or a sphingolipid-reducing agent.

In some embodiments, a lipid raft (e.g. caveolae) activity inhibitor is administered to prevent or treat the intoxicating effects of BoNT. Primarily, there are three main types of BoNT intoxifications: food borne, infant and wound botulism. And unfortunately, there is a fourth type of BoNT intoxification: deliberate release of BoNT. Foodborne botulism occurs when a person ingests pre-formed toxin that leads to illness within a few hours to days. Foodborne botulism is a public health emergency because the contaminated food may still be available to other persons besides the patient. With foodborne botulism, symptoms begin within 6 hours to 2 weeks (most commonly between 12 and 36 hours) after eating toxin-containing food. Symptoms of botulism include double vision, blurred vision, drooping eyelids, slurred speech, difficulty swallowing, dry mouth, muscle weakness that always descends through the body: first shoulders are affected, then upper arms, lower arms, thighs, calves, etc. Paralysis of breathing muscles can cause a person to stop breathing and die, unless assistance with breathing (mechanical ventilation) is provided. Infant botulism occurs in a small number of susceptible infants each year who harbor C. botulinum in their intestinal tract. Wound botulism occurs when wounds are infected with C. botulinum that secretes the toxin. Deliberate bioterror BoNT intoxification may have the following features: outbreak of a large number of cases of acute flaccid paralysis with prominent bulbar palsies; outbreak with an unusual botulinum toxin type (e.g., types C, D, F, G or E toxins which are not acquired from an aquatic food; outbreak with a common geographic factor among cases (e.g., airport) but without a common dietary exposure (e.g., features suggestive of an aerosol attack); and multiple simultaneous outbreaks with no common source.

Currently, a pentavalent vaccine that protects against active BoNT serotypes A-E and a separate monovalent vaccine that protects against active BoNT serotype F are available as Investigational New Drugs. However, there are numerous shortcomings associated with the toxoid vaccines. For example, serious adverse response to the antitoxins, such as anaphylaxis, has been reported to occur in 2% of recipients.

Other methods of combating botulinum intoxication are under investigation—most of which involve the administration of an antigen for the production of antibodies against the toxin. For example, Simpson et al. reports an inactive BoNT that may be administered orally to stimulate production of antibody in a mammal. See U.S. Pat. No. 6,051,239, the disclosure of which is incorporated in its entirety herein by reference. These methods which rely on the production of antibodies are not very practical because they require the mammal to be vaccinated before becoming intoxicated with the toxin. For example, if a non-vaccinated mammal is intoxicated with botulinum toxin, the administration of an antigen (e.g., an inactive BoNT) to stimulate antibodies production against the active BoNT is futile because the production of antibodies by the mammal would not be timely enough to ward off the deleterious effects of active BoNT, which occur within about 12 to 72 hours.

In some embodiments, an antibody directed against a caveolin is administered to prevent or treat botulinum toxin intoxication.

In some embodiments, a caveolin conjugate discussed above is administered to prevent or treat botulinum toxin intoxication.

An ordinarily skilled medical provider can determine the appropriate dose and frequency of administration(s) to achieve an optimum clinical result. That is, one of ordinary skill in medicine would be able to administer the appropriate amount of the lipid raft activity inhibitor at the appropriate time(s) to effectively prevent or treat botulinum toxin intoxication.

Moreover, an ordinarily skilled medical provider can determine the appropriate dose and frequency of administration(s) lipid raft activity inhibitor to achieve an optimum clinical result. That is, one of ordinary skill in medicine would be able to administer the appropriate amount of the lipid raft (e.g., caveolae) activity inhibitor at the appropriate time(s) to effectively prevent or treat Clostridial toxin intoxication.

In some embodiments, the mammal being treated is additionally subjected to close respiratory monitoring and feeding by enteral tube or parenteral nutrition, intensive care, mechanical ventilation, and/or treatment of secondary infections.

III. Methods of preventing or treating a metabolic disorder, a muscular condition, a nervous system disorder and/or a pain condition in a mammal: It is known that a Clostridial toxin, for example botulinum toxin, may be administered to treat a metabolic disorder, a muscular condition, a nervous system disorder and/or a pain condition in a mammal. The present invention improves upon this knowledge by combining the administration of a Clostridial toxin with a lipid raft activity enhancer to enhance the effect of the therapeutic Clostridial toxin.

In some embodiments, the method comprises the step of co-administering a lipid raft activity enhancer and a Clostridial toxin. Co-administering includes the administration of the lipid raft activity enhancer and Clostridial toxin simultaneously or sequentially (in any order).

Non-limiting examples of metabolic disorders include diabetes, obesity and hypertension. Non-limiting examples of muscular conditions include muscular dystrophy, strabismus, blepharospasm, spasmodic torticollis, oromandibular dystonia, and spasmodic dysphonia. A non-limiting example of a nervous system disorder include Alzheimer's disease. In some embodiments, the nervous system disorder can also be an autonomic nervous system disorder. Non-limiting examples of autonomic nervous system disorders are rhinorrhea, otitis media, excessive salivation, asthma, chronic obstructive pulmonary disease (COPD), excessive stomach acid secretion, spastic colitis, and excessive sweating. Non-limiting examples of pain conditions include migrane headaches, muscle spasm, vascular disturbances, angina, neuralgia, fibromyalgia, neuropathy, and pain associated with inflammation.

As described above, a lipid raft activity enhancer may be an antibody which links together (or causes colocalization or clustering of) components of lipid rafts; a lipid raft concentration enhancer; a caveolae activator, such as okadaic acid.

In some embodiments, the method comprises the step of co-administering a lipid raft enhancer and a botulinum toxin, for example botulinum toxin type A. In some embodiments, the lipid raft enhancer employed may be an antibody which colocalizes. In some embodiments, the lipid raft enhancer may be a caveolae activator, such as an okadaic acid. In some embodiment, an okadiac acid and a botulinum toxin type A is administered to prevent or treat diseases associated with lipid rafts.

An ordinarily skilled medical provider can determine the appropriate dose and frequency of administration(s) to achieve an optimum clinical result. That is, one of ordinary skill in medicine would be able to administer the appropriate amount of the lipid raft activity enhancer at the appropriate time(s) to effectively prevent or treat a metabolic disorder, a muscular condition, a nervous system disorder and/or a pain condition in a mammal.

An ordinarily skilled medical provider can determine the appropriate dose and frequency of administration(s) lipid raft activity enhancer to achieve an optimum clinical result. That is, one of ordinary skill in medicine would be able to administer the appropriate amount of the lipid raft (e.g., caveolae) activity inhibitor at the appropriate time(s) to effectively prevent or treat a metabolic disorder, a muscular condition, a nervous system disorder and/or a pain condition in a mammal.

IV. Methods of inhibiting the formation of lipid rafts (e.g., caveolae) on a cell membrane: Lipid rafts or caveolae formation may be inhibited by a Clostridial toxin. In some embodiments, the methods comprise the step of contacting the cell with a Clostridial toxin. As described above, the lipid rafts (e.g. caveolae) may be caveolin-containing lipid rafts or non-caveolin-containing lipid rafts. Without wishing to limit the invention to any theory or mechanism of operation, it is believed that the Clostridial toxin interacts with a caveolin protein, or other lipid raft component inside the cell. Caveolin proteins that may interact with a Clostridial toxin may include caveolin-1alpha, caveolin-1beta, caveolin-2, caveolin-3. Presumably, the caveolin proteins interact with the Clostridial toxin via a caveolin-interacting motif on the Clostridial toxin.

It is further believed that the interaction of the Clostridial toxin (e.g., botulinum toxin) with the caveolin brings the Clostridial toxin close to the vicinity of a Clostridial toxin substrate (e.g., SNAP 25, VAMP, etc) for the Clostridial toxin to enzymatically cleave the substrate. The cleavage of these substrates may prevent the formation of lipid rafts (e.g., caveolae). Also, the cleavage of these substrates may also prevent vesicle membrane fusion, and thereby inhibits the formation of new lipid rafts (e.g., caveolae) in the plasma membrane.

In some instances, the cleavage of these substrates may even disrupt existing lipid rafts. For example, the interaction of a Clostridial toxin with a caveolin protein within the context of a lipid raft may result in a conformational change of the toxin protein and/or lipid raft such that the toxin can pass through the membrane bilayer or itself form a pore in the bilayer and enter the cytoplasm. Once inside the cytoplasm, the endopeptidase activity of the botulinum toxin has access to its substrates such as SNAP-25 (or SNAP-23, the ubiquitously expressed analogue of neuronal SNAP-25) and VAMP proteins. Once cleaved, these substrates of botulinum toxin are no longer able to mediate vesicle fusion and any further exocytic or endocytic vesicle fusion events are disrupted. The resultant disruption of fusion of exocytic neurotransmitter-containing vesicles with the plasma membrane may ultimately result in a reduction in the formation, assembly or presence of new lipid rafts in the plasma membrane. Similarly, the degradation of SNAP and SNARE complex proteins within the cytoplasm may result in the disruption of existing endocytic lipid rafts if the SNAP and/or SNARE components on the cytoplasmic face of the membrane are required for the maintenance of a localized concentration of unique components within lipid rafts.

In some embodiments, a caveolin conjugate comprising a caveolin and an active Clostridial toxin may be employed to inhibit the formation of lipid rafts. For example, a caveolin conjugate comprising a caveolin-1 (or -2 or -3) and an active botulinum toxin (e.g., type A) may be administered to inhibit the formation of lipid rafts on a cell. In some embodiments, a caveolin conjugate comprising a flottillin and an active Clostridial toxin may be employed to inhibit the formation of lipid rafts. For example, a caveolin conjugate comprising a flottillin-1 (or -2) and an active botulinum toxin (e.g., type A) may be administered to inhibit the formation of lipid rafts on a cell.

V. Clostridial toxin chimeras: The present invention also provides for Clostridial toxin chimeras that are effective for use in treating diseases associated with lipid rafts. In some embodiments, the chimeras comprise a targeting moiety, a caveolin (or a flottillin), and a Clostridial toxin. The targeting moiety may bind to a receptor of a specific cell type, thus facilitating the entry of the Clostridial toxin into that cell. Without wishing to limit the invention to any theory or mechanism of operation, it is believed that once the Clostridial toxin is inside the cell, the caveolin brings the chimera to the cellular assemblies that form lipid rafts. Once the chimera is within the cellular assemblies that form the lipid rafts, the chimera is believed to enzymatically act on a Clostridial toxin substrate (e.g., SNAP 25, VAMP, etc.). The enzymatic actions of the chimera may result in the inhibition of the formation of a lipid raft or a caveolae. In some embodiments, the inhibition of the formation of lipid rafts or caveolae in certain cells is effective in treating diseases associated with lipid raft or caveolae formation.

In some embodiments, the targeting moiety and the caveolin are covalently linked to the Clostridial toxin using chemical techniques commonly known in the art. For example, see Example 16 and U.S. Pat. No. 6,203,794 to Dolly et al., the disclosure of which is incorporated in its entirety by reference herein. In some embodiments, the targeting moiety, the caveolin and the Clostridial toxin are expressed as a fusion protein, using techniques know to one of ordinary skill in the art.

The chimeras of the present invention include chimeras that have a targeting moiety (e.g., the targeting moieties discussed herein)/botulinum toxins (e.g., type A)/caveolin (or flottillin).

VI. Methods of treating a disease associated with a lipid raft or caveolae formation: Diseases associated with a formation of a lipid raft or a caveolae are diseases wherein the inhibition of lipid raft formation or inhibition of caveolae formation would alleviate the symptoms of the disease or treat the disease. There are various molecular bases for inhibiting the formation of lipid rafts to treat a disease. For example, lipid rafts or caveolae formations may play a role in the fusion of intracellular vesicles. An inhibition of a lipid raft formation would result in the inhibition of a vesicle fusion. The inhibition of vesicle fusion decreases the release of certain molecules, wherein the decrease in release of such molecules result in the treatment of certain diseases.

For example, hepatic insulin resistance, obesity and diabetes are diseases associated with lipid raft or caveolae formation. Adipocytes secrete several proteins known as adipocytokines which influence insulin sensitivity and glucose metabolism profoundly. These adipocytokines provide a molecular link between increased adiposity and impaired insulin sensitivity. It appears that a novel family of fat- and gut-derived circulating proteins modulates hepatic insulin action. For example, resistin is a member of the recently defined family of small cysteine-rich secreted proteins dubbed the resistin-like molecule family of hormones secreted by adipose tissue. Two other members of the family, resistin-like molecule-RELM (also known as FIZZ 1) and RELM-beta (also known as FIZZ2), are about 60% similar to resistin and are expressed in the stromal components of lung and adipose tissue and in epithelial cells of the intestine, respectively. This family of circulating proteins is likely to play a role in the complex interorgan communication network, which appears to modulate intermediate metabolism and energy balance. For example, it has been reported that the infusion of either resistin or the resistin-like molecule-beta (RELM-beta) rapidly induced severe hepatic insulin resistance.

Thus, hepatic insulin-resistance, obesity and diabetes may be treated by decreasing the secreted resistins from adipocytes or gut cells. In one embodiment, a chimera comprising a botulinum toxin/caveolin/targeting moiety directed to adipocytes or gut cells may be administered to decrease the release of resistins. The targeting moiety directs the chimera specifically to adipocytes. For example, targeting moieties in accordance with the present invention include peptide or protein ligands for cellular receptors, small molecules, and antibodies to cell type specific receptors or lipid raft components.

Examples of peptide ligands that may be used as targeting moieties are phosphoinositolglycans (PIG) and PIG-peptides (reported to activate the insulin receptor-independent insulin signaling cascade in adipocytes), a synthetic thrombin receptor peptide Ser-Phe-Phe-Leu-Arg-Asn-Pro (SFFLRNP) (which mimics the amino-terminus of thrombin receptor proteolytically activated by thrombin), and a soluble integrin-binding sequence peptide LDGGCRGDMFGCA (to target Mast cell integrin). Examples of protein ligands that may be used as targeting moieties are the glucose transporter GLUT4 (for which efficient endocytosis and association with the cell surface membrane of adipocytes is reported to influenced by caveolin), interleukin-4 (IL-4) and human IgE. Examples of small molecules that may be used as targeting moieties are the beta3-selective adrenergic receptor ligand BRL 37344, and the benzoylthiophene analog, PD 81,723 (an adenosine A(1) receptor allosteric enhancer for targeting to brain and adipocyte membranes). Examples of antibodies that may be used as targeting moieties are mAb UA009 which recognizes CD36/fatty acid translocase in adipocytes, and the mast-cell specific monoclonal antibody mAb AA4.

In some embodiments, a chimera that may be employed to prevent or treat hepatic insulin resistance, obesity and diabetes include a chimera comprising, for example, PIG/botulinum toxin/caveolin; or mAb UA009/botulinum toxin/caveolin; or SFFLRNP/botulinum toxin/caveolin.

Without wishing to limit the invention to any theory or mechanism of operation, it is believed that once the chimera is internalized into the adipocyte, the caveolin directs the chimera to the lipid raft assemblies associated with vesicle fusions, where a botulinum toxin substrate (e.g., SNAP) is also located. It is further believed that the botulinum toxin enzymatically cleaves these substrates, and thereby inhibit the vesicle fusions, which results in a decrease of release of resistins. In some embodiments, the chimera may be administered in conjunction with thiazolidinediones (which are believed to decrease insulin resistance via modulation of adipocytokine expression and are currently being used clinically in the treatment of Type 2 diabetes).

There are other molecular bases for inhibiting lipid rafts or caveolae to treat certain diseases. For example, lipid rafts or caveolae play a role in bringing certain proteins to the cell surface. The presentation of these specific proteins result in various medical conditions. The inhibition of lipid raft or caveolae would result in the decrease of these specific proteins at the cell surface. Thus, the inhibition of lipid raft or caveolae would also result in the treatment of various diseases.

For example, inflammation, infection and/or allergy may be treated by inhibiting the formation of lipid raft or caveolae. Caveolae are involved in bacterial (an antigen) entry into mast cells. The detection of caveolae in the microvilli and intracellular vesicles of hematopoietic cells (cultured mouse bone marrow-derived mast cells (BMMCs)) was recently reported. CD48, a receptor for type 1 fimbriated Escherichia coli, was specifically localized to caveolae in BMMCs. The involvement of caveolae in bacterial entry into BMMCs was demonstrated through the use of caveolae-disrupting and -usurping agents which specifically blocked E. coli entry, and markers of caveolae were actively recruited to sites of bacterial entry. Thus, it is believed that the formation of bacteria-encapsulating caveolar chambers in BMMCs represents a distinct mechanism of microbial entry into phagocytes.

Caveolae also appear to be involved in the synthesis of prostaglandins by immunoinflammatory cells. Group V secretory phospholipase A2 (PLA2), Group IV cytosolic PLA2 and cyclooxygenase-2 (COX-2) are key enzymes for arachidonic acid (AA) mobilization and prostaglandin (PG) production by cells such as macrophages and mast cells. Because Group V PLA2 is a secreted enzyme, it has been assumed that it must then reassociate with the outer membrane to release AA. It has been demonstrated that chronic exposure of the macrophages to lipopolysaccharide results in Group V PLA2 association with caveolin-2-containing granules close to the perinuclear region. Heparin blocks that association, suggesting that the granules are formed by internalization of the Group V sPLA2 previously associated with the outer cellular surface. As Group IV PLA2 and COX-2 are localized in the perinuclear region during cell activation, this process appears to bring Group V PLA2 to the perinuclear region which is closer to COX-2, where, if active, would have the potential for efficient prostaglandin synthesis.

To prevent or treat hematopoietic or immunoinflammatory conditions in a mammal, an effective amount of botulinum toxin may be administered to the mammal. It is believed that botulinum toxin is effective to inhibit the formation of caveolae on a cell membrane. Since the binding of antigen to mast cells and synthesis of prostaglandin depend on the caveolae formed on the mast cells, the inhibition of caveolae formation by botulinum toxin would effectively result in the reduction of binding of antigen to mast cells and reduction of synthesis of prostaglandin. Accordingly, the inflammation, infection and/or allergy conditions may be prevented or treated by the administration of botulinum toxin.

In some embodiments, the botulinum toxin may be more specifically directed to the mast cells by conjugating a botulinum toxin with a targeting moiety, forming a toxin conjugate. The targeting moiety specifically binds to receptors that are mainly found on the surface of mast cells. Examples of such targeting moieties include the soluble integrin-binding sequence peptide LDGGCRGDMFGCA (to target Mast cell integrin), the receptor for interleukin-4 (IL-4) and the human high-affinity IgE receptor Fc epsilon R.

In some embodiments, a chimera that may be employed for treating inflammaotry diseases include a chimera comprising, for example, LDGGCRGDMFGCA/botulinum toxin/caveolin.

An ordinarily skilled medical provider can determine the appropriate dose and frequency of administration(s) to achieve an optimum clinical result. That is, one of ordinary skill in medicine would be able to administer the appropriate amount of the chimera at the appropriate time(s) to effectively prevent or treat a disease associated with a lipid raft or caveolae formation.

An ordinarily skilled medical provider can determine the appropriate dose and frequency of administration(s) lipid raft activity enhancer to achieve an optimum clinical result. That is, one of ordinary skill in medicine would be able to administer the appropriate amount of the chimera at the appropriate time(s) to effectively prevent or treat a disease associated with a lipid raft of caveolae formation.

VII. Methods of identifying a compound that may alter the internalization of a Clostridial toxin: Compound that alter the internalization of Clostridial toxins may be screened. In some embodiments, the methods comprise the step of contacting a test compound with a cell that is can internalize Clostridial toxins. The internalization of Clostridial toxin by this cell is compared with a cell that is contacted by a negative control compound. If the cellular internalization of Clostridial toxin is enhanced by the contacting with the test compound (as compared to the negative control), then the test compound is an internalization enhancer. If the cellular internalization of Clostridial toxin is inhibited by the contacting with the test compound, then the test compound is an internalization inhibitor.

Although examples of routes of administration and dosage are provided for the methods of treatment inventions herein, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14^{th} edition, published by McGraw Hill).

The present invention also includes formulations which comprise at least one of the compositions disclosed herein, e.g, lipid raft activity enhancers, lipid raft activity inhibitors, chimeras, and combinations thereof. In some embodiments, the formulations comprise at least one of the compositions disclosed herein in a pharmacologically acceptable carrier, such as sterile physiological saline, sterile saline with 0.1% gelatin, or sterile saline with 1.0 mg/ml bovine serum albumin.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Method of Inhibiting the Formation of Lipid Rafts on a Cell: Use of Botulinum Toxin to Prevent or Treat Atherosclerosis

The development of atherosclerosis is a process characterized by the accumulation of lipids in the form of modified lipoproteins in the subendothelial space. This initiating step is followed by the subsequent recruitment and proliferation of other cell types, including monocytes/macrophages and smooth muscle cells. Caveolin-1 is a principal structural protein component of caveolae membrane domains, and caveolae are involved in the pathogenesis of atherosclerosis. It has been reported that, in mice, loss of caveolin-1 in an ApoE−/− background resulted in a dramatic increase in non-HDL plasma cholesterol levels. However, despite this hypercholesterolemia, the loss of caveolin-1 gene expression was clearly protective against the development of aortic atheromas, with up to an approximately 70% reduction in atherosclerotic lesion area. Loss of caveolin-1 resulted in the dramatic downregulation of certain proatherogenic molecules, namely, CD36 and vascular cell adhesion molecule-1. Thus, loss of caveolin-1 can counteract the detrimental effects of atherogenic lipoproteins.

At this point, it is unclear whether the reduction in caveolin-1 or reduction in caveolae results in down regulation of certain proatherogenic molecules. Regardless, the administration of botulinum toxin is effective in inhibiting the formation of caveolae, and consequently the concentration of caveolin on the cell surface. Thus, a therapeutically effective amount of botulinum toxin may be administered to endothelial cells to reduce the accumulation atherosclerotic lesions.

### Example 2

### Method of Inhibiting the Formation of Lipid Rafts Associated with Vesicle Fusion: Use of a Chimera to Prevent Tumorigenesis

Mammary epithelial cells are embedded in a unique extracellular environment to which adipocytes and other stromal cells contribute, and are dependent on this milieu for survival. Adipocytokines are reported to uniquely influence the characteristics and phenotypic behavior of malignant breast ductal epithelial cells; adipocyte-secreted factors promote mammary tumorigenesis through induction of anti-apoptotic transcriptional programs and proto-oncogene stabilization. Adipocytokines specifically induce several transcriptional programs involved in promoting tumorigenesis, including increased cell proliferation, invasive potential, survival, and angiogenesis.

Regulation of the levels of adipocytokines in breast ductal epithelial cells may lead to a reduction in the tumorigenic potential, proliferation, invasiveness, immortality, and angiogenic potential associated with oncogenic transformation.

A chimera comprising a caveolin/botulinum toxin/targeting moiety directed to adipocytes or breast ductal epithial cells or other stromal cells to reduce the secretion of adipocytokines may be employed to treat cancer. The targeting moiety directs the chimera specifically to adipocytes, ductal epithial cells or other stromal cells. For example, targeting moieties include peptide or protein ligands for cellular receptors, small molecules, and antibodies to cell type specific receptors or lipid raft components. Examples of peptide ligands that may be used as targeting moieties are phosphoinositolglycans (PIG) and PIG-peptides (reported to activate the insulin receptor-independent insulin signaling cascade in adipocytes), a synthetic thrombin receptor peptide Ser-Phe-Phe-Leu-Arg-Asn-Pro (SFFLRNP) (which mimics the amino-terminus of thrombin receptor proteolytically activated by thrombin), and a soluble integrin-binding sequence peptide LDGGCRGDMFGCA (to target Mast cell integrin). Examples of protein ligands that may be used as targeting moieties are the glucose transporter GLUT4 (for which efficient endocytosis and association with the cell surface membrane of adipocytes is reported to influenced by caveolin), interleukin-4 (IL-4) and human IgE. Examples of small molecules that may be used as targeting moieties are the beta3-selective adrenergic receptor ligand BRL 37344, and the benzoylthiophene analog, PD 81,723 (an adenosine A(1) receptor allosteric enhancer for targeting to brain and adipocyte membranes). Examples of antibodies that may be used as targeting moieties are mAb UA009 which recognizes CD36/fatty acid translocase in adipocytes, and the mast-cell specific monoclonal antibody mAb AA4. Without wishing to limit the invention to any theory or mechanism of operation, it is believed that once the chimera is internalized into these cells, e.g., the adipocyte, the caveolin directs the chimera to the lipid raft assemblies associated with vesicle fusions, where a botulinum toxin substrate (e.g., SNAP) is also located. It is further believed that the botulinum toxin enzymatically cleaves these substrates, and thereby inhibit the vesicle fusions, which results in a decrease of release of adipocytokines. Thus, the chimera of the present invention may mediate the reduction of secretion of adipocytokines and thereby alter the phenotypic behavior of malignant breast ductal epithelial cells, reduce their tumorigenic potential and metastasis, and thereby provide a means of treating breast cancer.

In some embodiments, a chimera of the present invention may be administered in conjunction with other anticancer agents (such as taxol or tamoxifen).

### Example 3

### Method of Inhibiting the Formation of Lipid Rafts: Use of Lipid Raft Formation Inhibitor (e.g., Lipid Raft Activity Inhibitor or Botulinum Toxin) to Treat Alhzeimer's Disease

It is known that the amyloid precursor protein (APP) is a precursor of beta-amyloid (A-beta) peptide, the principal protein component found in senile plaques within the brains of patients with Alzheimer's disease. Two competing proteolytic pathways play a key role in the etiology of Alzheimer's disease. In the first, A-beta peptide is generated from APP by the beta- and gamma-secretases. In the alternative pathway, alpha-secretase cleaves APP within the A-beta amino acid sequence, thereby precluding the formation of A-beta peptide. Thus, enhancing the proteolysis of APP by alpha-secretase or reducing the proteolysis of APP by beta- and gamma-secretases in neural tissue is advantageous in combating Alzheimer's disease.

Caveolin proteins have been proposed to play a key role in APP processing (Engelman, et al., 1998 Am. J. Hum. Genet. 63:1578-87). It is known that lipid rafts from whole brain contain APP as well as A-beta peptide (Lee, et al., 1998 Nat. Med. 4:730-34), and caveolae are believed to be sites of enrichment of APP, providing a direct means for APP to be concentrated. It has been reported that overexpression of recombinant caveolin-1 protein promoted alpha-secretase-mediated cleavage of APP, and that, conversely, this proteolysis of APP was abolished by blocking caveolin-1 expression using antisense oligonucleotides (Ikezu, et al., 1998 J. Bio. Chem. 273:10485-95). Thus, increasing caveolin-1 concentration or activity in lipid rafts promotes alpha-secretase-mediated cleavage of APP and prevents formation of A-beta peptide.

It has also been reported that a reduction in lipid rafts efficiently inhibits A-beta peptide secretion in cultured hippocampal neurons (Simons, et al., 1998 PNAS 95:6460-64). Thus, some embodiments, a patient who is a candidate for or is suffering from Alzheimer's Disease may be treated by the administration of a lipid raft activity inhibitor. As discussed, a Clostridial toxin may also inhibit the formation of lipid raft. Thus, in some embodiments, a patient who is a candidate for or is suffering from Alzheimer's Disease may be treated by the administration of a botulinum toxin.

### Example 4

### Exemplary Methods for Treatment of Pain Associated with Muscle Disorder with BoNT and a Lipid Raft Activity Enhancer

An unfortunate 36 year old woman has a 15 year history of temporomandibular joint disease and chronic pain along the masseter and temporalis muscles. Fifteen years prior to evaluation she noted increased immobility of the jaw associated with pain and jaw opening and closing and tenderness along each side of her face. The left side is originally thought to be worse than the right. She is diagnosed as having temporomandibular joint (TMJ) dysfunction with subluxation of the joint and is treated with surgical orthoplasty meniscusectomy and condyle resection.

She continues to have difficulty with opening and closing her jaw after the surgical procedures and for this reason, several years later, a surgical procedure to replace prosthetic joints on both sides is performed. After the surgical procedure progressive spasms and deviation of the jaw ensues. Further surgical revision is performed subsequent to the original operation to correct prosthetic joint loosening. The jaw continues to exhibit considerable pain and immobility after these surgical procedures. The TMJ remained tender as well as the muscle itself. There are tender points over the temporomandibular joint as well as increased tone in the entire muscle. She is diagnosed as having post-surgical myofascial pain syndrome and is injected with 7 U/kg of the BoNT (preferably type A) and a therapeutically effective amount of lipid raft activity enhancer into the masseter and temporalis muscles.

Several days after the injections she noted substantial improvement in her pain and reports that her jaw feels looser. This gradually improves over a 2 to 3 week period in which she notes increased ability to open the jaw and diminishing pain. The patient states that the pain is better than at any time in the last 4 years. The improved condition persists for up to 27 months after the original injection of the modified neurotoxin.

### Example 5

### Accidental Overdose in the Treatment of Postherpetic Neuralgia—Use of Lipid Raft Activity Inhibitor as an Antidote

The anaerobic, gram positive bacterium Clostridium botulinum produces a potent polypeptide neurotoxin, botulinum toxin. Botulinum toxin causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex) (Available from Allergan, Inc., of Irvine, Calif. under the tradename BOTOX® in 100 unit vials) is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each.

Postherpetic neuralgia is one of the most intractable of chronic pain problems. Patients suffering this excruciatingly painful process often are elderly, have debilitating disease, and are not suitable for major interventional procedures. The diagnosis is readily made by the appearance of the healed lesions of herpes and by the patient's history. The pain is intense and emotionally distressing. Postherpetic neuralgia may occur any where, but is most often in the thorax.

In an exemplary scenario, a 76 year old man presents a postherpetic type pain. The pain is localized to the abdomen region. The patient is treated by a bolus injection of between about 0.05 U/kg to about 2 U/kg of a BOTOX® intradermally to the abdomen. The treating physician accidentally administers an excessive amount of BOTOX®. Upon realizing the error, the physician administers the same area with a therapeutically effective dose of lipid raft activity inhibitor. The particular dose as well as the frequency of administrations g-iBoNT depends upon a variety of factors within the skill of the treating physician. Within 1-7 days after BOTOX® and corrective g-iBoNT administration, the patient's pain is substantially alleviated.

### Example 6

### Detoxification with Lipid Raft Activity Inhibitor

Aerosol distribution of a BoNT can result in symptoms of botulism. For example. A pentavalent (ABCDE) botulinum toxoid is available from the Centers for Disease Control and Prevention, but its use may not be feasible as a prophylaxis due to the need to wait for antibodies to be raised in the recipient before immunity can be conferred.

Thus, in terms of detoxification or post exposure treatments, the toxoid is unfeasible because it induces immunity over several months. Immediate immunity can be provided by passive administration of equinine botulinum antitoxin or by specific human hyperimmune globulin. However, these means of detoxification are not very effective. For example, a segment of the population is known to suffer from horse serum anaphylaxis with the administration of the equinine botulinum antitoxin.

Lipid raft activity inhibitor can play a significant role in the detoxification of the individuals contaminated with an active BoNT. In a clinical or emergency setting, injection of victims with lipid raft activity inhibitor could provide enough inhibition of transport of the toxin into the cells to minimize its effects. In some embodiments, lipid raft activity inhibitors may be formulated in pills to allow safe, quick and easy access for a large patient population.

### Example 7

### Exemplary Methods of Making a Chimera (Botulinum Toxin/Targeting Moiety/Caveolin)

It is known that most molecules acting as substrates or binding molecules, such as the targeting moiety, have positions that are not sensitive to steric hindrance. In addition, the linkage process should not introduce chirality into the targeting moiety. Further, the linker and the targeting moiety should be attached through a covalent bond. The distance between the Bot and the targeting moiety may be adjusted by the insertion of spacer components. Preferable spacers have functional groups capable of binding to the linker, targeting moiety and Bot and serving to conjugate them. Preferred spacer components include:
1) HOOC—(CH₂)ₙ—COOH, where n=1-12, suitable for insertion at the amino terminal end of a peptide, to connect it with a linker on a targeting moiety.
2) HO—(CH₂)ₙ—COOH, where n>10, suitable for attachment at the amino terminal of a peptide to connect the L chain with a linker on a targeting moiety.
3) (C₅H₆)ₙ, where n>2, suitable for attachment to join the Bot with a linker on the targeting moiety. The benzene rings provide a rigid spacer between the targeting moiety and Bot. Of course, appropriate functional groups, for example as identified by X below, will be present on the benzene rings to link the drug and the Bot.

Various linker types are envisioned. For example, in one type the targeting moiety-linker-Bot molecule remains intact after introduction into the circulatory system.

In some embodiments, a cysteine residue is attached to the end of the Bot molecule by methods well known in the art. For instance, the gene construct that expresses the Bot protein can be mutated to express a cysteine residing at the N-terminal portion of the protein. A maleimide linker is then attached to the Cysteine residue by well known means.

In some embodiments, the linker is attached directly to the targeting moiety. A targeting moiety-X moiety can have the following groups wherein X may be, without limitation, OH, SH, NH₂, CONH, CONH₂, COOH, COOR₃₀ (where R₃₀ is an alkyl group). Of course, the proper group would not be in an active site or be sterically hindering. The following is an example of one reaction which would link the targeting moiety-X to the linker molecule.

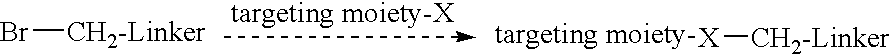

Once the targeting moiety has a linker attached, the following reaction can be used to link the targeting moiety to the Bot. In this reaction, the Bot, preferably the Bot has an accessible lysine group that is used as the attachment point for the targeting moiety. As discussed herein, an extra amino acid, such as lysine, can be readily added to the N-terminal portion of the Bot gene and used as the attachment point for a targeting moiety. In the following reaction, sodium cyanoborohydride is used to attach the linker to the lysine group on the Bot molecule.
targeting moiety-linker-CHO+NaCNBH₃+Bot-Lys→
targeting moiety-linker-CH₂—NH-Bot

Targeting moiety that are envisioned for use in the present invention include those that have a free —XH group and that can bind to liver and/or kidney transporters.

Once the Targeting moiety is linked to the Bot, similar techniques may be employed to link the targeting moiety-Bot to a caveolin to form a targeting moiety/Bot/caveolin chimera. See U.S. Pat. No. 6,203,794 to Dolly, the disclosure of which is incorporated in its entirety herein by reference.

### Example 8

### Exemplary Methods of Making a Conjugate Comprising an Inactive Botulinum Toxin as a Transporter

The method exemplified by Example 7 may be employed to create conjugates, for example, conjugates comprising a transporter. In some embodiments, the method of Example 7 is employed to create a conjugate comprising an antibody against a caveolin, an inactive botulinum toxin as a transporter. In some embodiment, the method of Example 7 is employed to create a conjugate comprising a caveolin and an active botulinum toxin.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method of altering the degree of internalization of a Clostridial toxin into a cell, said method comprises the step of:
altering an activity of a lipid raft on a membrane of a cell, thereby altering the degree of internalization of the Clostridial toxin into the cell.

2. The method of claim 1, wherein the lipid rafts are caveolae.

3. The method of claim 1, wherein the lipid rafts are selected from the group consisting of caveolin-containing lipid rafts and non-caveolin-containing lipid rafts.

4. The method of claim 3, wherein the caveolin-containing lipid rafts contain a caveolin family member selected from the group consisting of caveolin-1alpha, caveolin-1 beta, caveolin-2, caveolin-3, flottillin-1, flottillin-2 and combinations thereof.

5. The method of claim 4, wherein the caveolin family member is specifically expressed in a cell type selected from the group consisting of neuronal cells, astrocytes, glial cells, striated muscle cells, smooth muscle cells, cardiac cells, adipocytes, endothelial cells, secretory cells, type I pneumocytes, lung cells, kidney cells, dendritic cells, Mast cells, macrophages, T-cells, and B-cells.

6. The method of any of claims 1-5, wherein the activity lipid rafts is decreased by contacting the membrane of a cell with an activity inhibitor.

7. The method of claim 6, wherein the activity inhibitor comprises an antibody.

8. The method of claim 6, wherein the antibody is selected from the group consisting of humanized antibodies, polyclonal antibodies, monoclonal antibodies, and function blocking antibodies.

9. The method of claim 7, wherein the antibody is selected from the group consisting of antibodies against caveolin-1alpha, caveolin-1beta, caveolin-2, caveolin-3, flotillin-1, flotillin-2, reggie-1, reggie-2, stomatin, VIP36, LAT/PAG, MAL, BENE, syntaxin-1, syntaxin-4, synapsin I, adducin, VAMP2, VAMP/synaptobrevin, synaptobrevin II, SNARE proteins, SNAP-25, SNAP-23, a membrane-associated Clostridial toxin receptor protein, synaptotagrnin I, synaptotagmin II and GPI-anchored proteins.

10. The method of claim 7, wherein the antibody is selected from the group consisting of antibodies against GM1, GD1a, GD1b, GQ1b and GT1b.

11. The method of claim 1, wherein the activity is altered by changing the concentration of the lipid rafts.

12. The method of claim 11, wherein the activity of lipid rafts is decreased by contacting the membrane of a cell with a lipid raft concentration inhibitor.

13. The method of claim 12, wherein the lipid raft concentration inhibitor comprises a cholesterol-reducing agent.

14. The method of claim 13, wherein the cholesterol-reducing agent is selected from the group consisting of a statin, a cyclodextrin, a saponin, and a filipin.

15. The method of claim 12, wherein the lipid raft concentration inhibitor comprises a sphingolipid-reducing agent.

16. The method of claim 15, wherein the sphingolipid-reducing agent is a synthetic sphingolipid analogue.

17. The method of claim 15, wherein the sphingolipid-reducing agent is an inhibitor of sphingolipid synthesis.

18. The method of claim 17, wherein the inhibitor of sphingolipid synthesis is selected from the group consisting of L-cycloserine, fumonisin B1, and D-threo-1-phenyl-2-decanoylamino-3-morpholino-1-propanol.

19. The method of any of claims 1-5, wherein the activity of lipid rafts is increased by contacting the membrane of a cell with a lipid raft activity enhancer.

20. The method of claim 19, wherein the activity enhancer comprises an antibody.

21. The method of claim 20, wherein the antibody links together (or causes colocalization or clustering of) components of lipid rafts.

22. The method of claim 19, wherein the activity enhancer comprises a lipid raft concentration enhancer.

23. The method of claim 22, wherein the lipid raft concentration enhancer comprises a cholesterol-enhancing agent.

24. The method of claim 23, wherein the cholesterol-enhancing agent is a synthetic cholesterol analogue.

25. The method of claim 22, wherein the lipid raft concentration enhancer comprises a sphingolipid-enhancing agent.

26. The method of claim 25, wherein the sphingolipid-enhancing agent is a synthetic sphingolipid analogue.

27. A method of preventing or treating botulinum intoxication in a mammal, said method comprises the step of administering a lipid raft activity inhibitor, thereby preventing or treating botulinum intoxication.

28. The method of claim 27, wherein the lipid raft activity inhibitor comprises an antibody.

29. The method of claim 28, wherein the antibody is selected from the group consisting of humanized antibodies, polyclonal antibodies, monoclonal antibodies, and function blocking antibodies.

30. The method of claim 28, wherein the antibody is selected from the group consisting of antibodies against caveolin-1alpha, caveolin-1beta, caveolin-2, caveolin-3, flotillin-1, flotillin-2, reggie-1, reggie-2, stomatin, VIP36, LAT/PAG, MAL, BENE, syntaxin-1, syntaxin-4, synapsin I, adducin, VAMP2, VAMP/synaptobrevin, synaptobrevin II, SNARE proteins, SNAP-25, SNAP-23, a membrane-associated Clostridial toxin receptor protein, synaptotagmin I, synaptotagmin II and GPI-anchored proteins.

31. The method of claim 28, wherein the antibody is selected from the group consisting of antibodies against GM1, GD1a, GD1b, GQ1b and GT1b.

32. The method of claim 27, wherein the lipid raft activity inhibitor comprises a cholesterol-reducing agent.

33. The method of claim 32, wherein the cholesterol-reducing agent is selected from the group consisting of a statin, a cyclodextrin, a saponin, and a filipin.

34. The method of claim 27, wherein the lipid raft activity inhibitor comprises a sphingolipid-reducing agent.

35. The method of claim 34, wherein the sphingolipid-reducing agent is a synthetic sphingolipid analogue.

36. The method of claim 35, wherein the sphingolipid-reducing agent is an inhibitor of sphingolipid synthesis.

37. The method of claim 36, wherein the inhibitor of sphingolipid synthesis is selected from the group consisting of L-cycloserine, fumonisin B1, and D-threo-1-phenyl-2-decanoylamino-3-morpholino-1-propanol.

38. A method of preventing or treating a medical condition selected from a metabolic disorder, a muscular condition, a nervous system disorder and/or a pain condition in a mammal, said method comprises the step of administering a lipid raft activity enhancer, and administering a Clostridial toxin, thereby preventing or treating said metabolic disorder, muscular condition, nervous system disorder, pain and combinations thereof.

39. The method of claim 38, wherein said metabolic disorder is selected from the group consisting of diabetes, obesity and hypertension.

40. The method of claim 38, wherein said muscular condition is selected from the group consisting of muscular dystrophy, strabismus, blepharospasm, spasmodic torticollis, oromandibular dystonia, and spasmodic dysphonia.

41. The method of claim 38, wherein the nervous system disorder is an autonomic nervous system disorder.

42. The method of claim 41, wherein the autonomic nervous system disorder is selected from the group consisting of rhinorrhea, otitis media, excessive salivation, asthma, chronic obstructive pulmonary disease (COPD), excessive stomach acid secretion, spastic colitis, and excessive sweating.

43. The method of claim 38, wherein the pain condition is selected from the group consisting of migrane headaches, muscle spasm, vascular disturbances, angina, neuralgia, fibromyalgia, neuropathy, and pain associated with inflammation.

44. The method of claim 38, wherein the activity enhancer comprises an antibody.

45. The method of claim 44, wherein the antibody links together (or causes colocalization of) components of lipid rafts.

46. The method of claim 38, wherein the activity enhancer comprises a lipid raft concentration enhancer.

47. The method of claim 46, wherein the lipid raft concentration enhancer comprises a cholesterol-enhancing agent.

48. The method of claim 47, wherein the cholesterol-enhancing agent is a synthetic cholesterol analogue.

49. The method of claim 46, wherein the lipid raft concentration enhancer comprises a sphingolipid-enhancing agent.

50. The method of claim 49, wherein the sphingolipid-enhancing agent is a synthetic sphingolipid analogue.

51. The method of claim 38, wherein the lipid raft activity enhancer is a caveolae activator.

52. A method of inhibiting the formation of lipid rafts on a cell, said method comprising the step of contacting the cell with a Clostridial toxin, thereby inhibiting the formation of a lipid raft on a cell.

53. The method of claim 52, wherein the lipid rafts are caveolae.

54. The method of claim 52, wherein the lipid rafts are selected from the group consisting of caveolin-containing lipid rafts and non-caveolin-containing lipid rafts.

55. The method of claim 52, wherein the Clostridial toxin interacts with a caveolin family member.

56. The method of claim 55, wherein the caveolin family member is specifically expressed in one or more cell types selected from the group consisting of neuronal cells, astrocytes, glial cells, striated muscle cells, smooth muscle cells, cardiac cells, adipocytes, endothelial cells, secretory cells, type I pneumocytes, lung cells, kidney cells, dendritic cells, Mast cells, macrophages, T-cells, and B-cells.

57. The method of claim 55 or claim 56, wherein the caveolin family member is selected from the group consisting of caveolin-1alpha, caveolin-1beta, caveolin-2, caveolin-3, flottillin-1, flottillin-2 and combinations thereof.

58. A method of treating a disease associated with lipid rafts, said method comprising the step of administering a Clostridial toxin.

59. The method of claim 58 wherein the disease is selected from the group consisting of hepatic insulin resistance, obesity, diabetes, hematopoietic condition, immunoinflammatory condition, and Alzheimer's disease.

60. A method of identifying a compound that alters internalization of a Clostridial toxin into a cell, said method comprises the steps of:
contacting a cell sensitive to Clostridial toxin with a test compound, and screening for compounds that alter the affinity of the Clostridial toxin for lipid rafts.
